(19) United States
(12) Patent Application Publication
(10) Pub. No.: US 2009/0191620 A1
(43) Pub. Date: Jul. 30, 2009

(54) LIMITED ACCESS MULTI-LAYER CELL CULTURE SYSTEM

(76) Inventors: GREGORY R. MARTIN, Acton, ME (US); Allison J. Tanner, Portsmouth, NH (US)

Correspondence Address: CORNING INCORPORATED, SP-TI-3-1, CORNING, NY, 14831, US

(21) Appl. No.: 12/357,676

(22) Filed: Jan. 22, 2009

Related U.S. Application Data
(60) Provisional application No. 61/062,404, filed on Jan. 25, 2008.

(51) Int. Cl. C12M 1/00

(52) U.S. Cl. 435/294.1

(57) ABSTRACT

The present invention provides a multi-layer cell culture device having a rectangular footprint and having multiple cell culture chambers separated by tracheal air spaces, each cell culture chamber having a port and a port cover or an external manifold structured and arranged to allow for transfer of fluid into and out of the cell culture device with reduced risk of contamination, and methods of using the device.

### CROSS REFERENCE TO RELATED APPLICATIONS

This Application claims the benefit of U.S. Provisional Application Ser. No. 61/062,404 filed Jan. 25, 2008 and entitled “Limited Access Multi-Layer Cell Culture System”.

### FIELD

The present invention relates generally to a system for containing cells in culture. More specifically, the present invention relates to devices for containing cells in culture which allow for sterile controlled containment and sterile transfer of cells and media into and out of the device.

### BACKGROUND

In vitro culturing of cells provides material necessary for research in pharmacology, physiology, and toxicology. Recent advances in pharmaceutical screening techniques allow pharmaceutical companies to rapidly screen vast libraries of compounds against therapeutic targets. These large-scale screening techniques require large numbers of cells grown and maintained in vitro. Maintaining these large numbers of cells requires large volumes of cell growth media and reagents and large numbers and types of laboratory cell culture containers and laboratory equipment. This activity is also labor intensive.

Cells are grown in specialized cell culture containers including roller bottles, cell culture dishes and plates, multiwell plates, microtiter plates, common flasks and multi-layered cell growth flasks and vessels. Cells in culture attach to and grow on the bottom surface(s) of the flask, immersed in a suitable sustaining media.

With the advent of cell-based high throughput applications, cell culture vessels have been developed to provide an increased surface area for cell growth while also providing necessary gas exchange. These systems also employ traditional cell culture vessels including common flasks, roller bottles, cell culture dishes, as well as multi-layered cell growth vessels including multi-layer flasks, multi-layer cell culture dishes, bioreactors, cell culture bags and the like, which may include specialized surfaces designed to enhance the cell culture parameters including growth density and differentiation factors.

In addition, cell-based high throughput applications have become automated. Automation permits manipulation of the cell culture vessel much like that performed by the manual operator. Further, flask vessels having multiple layers of cell growth surfaces are capable of producing greater yields of adherent cells than commonly known flasks that permit growth of cells on a single bottom wall. While these multiple layer vessels allow for the growth of large numbers of cells, they present special challenges in day to day use.

There is a need for a cell culture vessel that can provide a device to direct fluid into and out of a cell culture vessel in a way that can be automated, and that improves the sterility of the transfer. In addition, there is a need for such a device that may be suitable for use in the performance of high throughput assay applications that commonly employ robotic manipulation.

### SUMMARY OF THE INVENTION

Embodiments of the present invention provide a multi-layer cell culture device having at least three cell culture chambers and at least two integral tracheal chambers, each cell culture chamber having at least one port, each port having a port cover where each port is structured and arranged to engage with a port cover to provide a releasable liquid tight seal. The port cover may be attached to the multi-layer cell culture device by a connector, which may be a hinged connector. In embodiments, the port cover may have a septum. The septum may allow for a fluid flow device, introduced through the septum, to form a liquid-tight seal between the fluid flow device and the port. In embodiments, the fluid flow device may be a needle, a pipette or pipette tip, a tube or a cannula. In embodiments, the port may have a sealer which can be an annular structure to allow a port cover to connect to a complimentary structure on port to form a reversible liquid-tight seal.

In additional embodiments, the present invention provides a cell culture device having at least one port and having a sliding port cover structured and arranged to engage with the at least one port to provide either an open or a closed port by slidingly engaging the port cover in an open position or a closed position in relation to the at least one port. In embodiments, the sliding port cover can be connected to the cell culture device by a connector such as a hinged connector, or the sliding port cover can be integral with the cell culture device.

In additional embodiments, the present invention provides a cell culture vessel having at least three rigid cell culture chambers, each cell culture chamber having at least one port; wherein the at least one port has a protruding male feature structured and arranged to couple with a female fluid flow device. In additional embodiments, cell culture vessel has at least one port having a female feature structured and arranged to couple with a male structure of a fluid flow device. In additional embodiments, the cell culture vessel has some ports with male structures and some ports with female structures to couple with complimentary structures of fluid flow devices.

In additional embodiments, the present invention provides a multi-layer cell culture device having at least three cell culture chambers and at least two integral tracheal chambers, each cell culture chamber having at least one port and a removable manifold structured and arranged to form a liquid-tight seal with the at least one port. In further embodiments, the manifold has a valve. In still further embodiments, the manifold is structured and arranged to couple with at least one port of more than one multi-layer cell culture device, or to couple one multi-layer cell culture device with a liquid reservoir.

In further embodiments, the present invention provides a manifold having fluid flow devices structured and arranged to engage with the ports of more than one multi-layer cell culture devices. Additionally, the present invention provides a cell culture system having at least one multi-layer cell culture device having at least two cell culture chambers, at least one external manifold having a manifold body and at least two fluid flow devices structured and arranged provide for the flow of fluid between the at least one external manifold and the at least two cell culture chambers of the multi-layer cell culture device; wherein fluid flows into the external manifold and is pooled in the manifold body before being distributed to the at least two fluid flow devices allowing fluid to flow between the at least one external manifold and the at least two cell culture chambers in parallel.

### BRIEF DESCRIPTION OF THE DRAWINGS

The invention is best understood from the following detailed description when read with the accompanying drawing figures.

FIG. 1 is a partial cut-away perspective view of an embodiment of the present invention.

FIGS. 2A, 2B and 2C are illustrations of embodiments of ports and port covers of the present invention.

FIG. 3 is an illustration of another embodiment of a port and sliding port cover of the present invention.

FIG. 4 is an illustration of three embodiments of the connector of the present invention.

FIGS. 5A and 5B are illustrations of embodiments of the sealers of the present invention.

FIG. 6 is an illustration of embodiments of the multi-layer cell culture flask of the present invention, illustrating ports and ports coupled to fluid flow devices of the present invention.

FIG. 7 is an illustration of embodiments of ports and port couplings of the present invention.

FIGS. 8A, 8B and 8C are illustrations of embodiments of the cannula of the present invention.

FIGS. 9A, 9B and 9C are illustrations of an embodiment of a manifold of the present invention, showing embodiments of valves of the present invention.

FIG. 10 is an illustration of an embodiment of the manifold of the present invention showing coupling of the fluid flow devices of the present invention to the manifold of the present invention.

FIGS. 11A and 11B are illustrations of an embodiment of the manifold of the present invention.

FIGS. 12A and 12B are illustrations of embodiments of the valve of the manifold of the present invention.

FIGS. 13A and 13B are additional illustrations of embodiments of the valve of the manifold of the present invention.

FIG. 14 is an illustration of an embodiment of the manifold of the present invention.

FIG. 15 is an additional illustration of an embodiment of the manifold of the present invention.

FIG. 16 is an additional illustration of an embodiment of the manifold of the present invention.

FIG. 17 is an illustration of a multi-layer flask of the present invention and its coupling with an embodiment of a manifold of the present invention.

FIG. 18 is an illustration of two multi-layer flasks of the present invention and their coupling with an embodiment of a manifold of the present invention.

FIG. 19 is an illustration of a multi-layer flask of the present invention and its coupling with an embodiment of a manifold of the present invention.

FIG. 20 is a perspective view of an embodiment of the present invention showing an embodiment of the manifold of the present invention linked to a container.

### DETAILED DESCRIPTION

Embodiments of the present invention relate to a limited access cell culture system and device. In embodiments, the limited access cell culture device of the present invention has multiple layers of cell growth chambers in an integral multi-layer cell culture device, each layer having a port, reversibly sealable with a port cover, to allow for the introduction and removal of material into and out of the cell growth chamber. In embodiments, the device also has an external manifold to control the flow of fluid into and out of the cell growth chamber.

In the following detailed description, for purposes of explanation and not limitation, exemplary embodiments disclosing specific details are set forth in order to provide a thorough understanding of the present invention. However, it will be apparent to one having ordinary skill in the art that the present invention may be practiced in other embodiments that depart from the specific details disclosed herein. In other instances, detailed descriptions of well-known devices and methods may be omitted so as not to obscure the description of the present invention.

Increasingly, cell cultures, particularly adherent cell cultures, are grown in stacked, space saving high density containers which minimize incubator space and maximize cell culture growth surface. See, for example, US Publication No. 2007/0026516. As cell culture containers become more and more efficient, and the spaces within them become more and more restricted, the practical use of these containers becomes complicated by the need to move small quantities of liquids into and out of these containers.

Maintaining the sterility of these high density containers and the fluid and cells contained within them is of the utmost importance. For example, vessels used to expand and treat cells in culture require that the cells be grown in a sterile system. One way to optimize the sterility of a cell culture container is to provide a closed or limited access cell culture system. A closed system may maintain the integrity of the cells in culture and prevent contamination. Cells in culture for therapeutic use may be unique to an individual, and may require conditions to promote proliferation and specific medical treatment without contamination. Cell culture vessels may have caps that are alternately removed and applied for access into the cell culture vessel, for example to add or remove cells or culture media. Or, alternatively, cell culture vessels may have a septum instead of cap. Culture contamination can result from material pushed into the vessel from the outside as the septum is punctured. The risk of contamination can be minimized by minimizing access to the cell culture chambers. For example, if a cell culture container is sterilized, and all of the interconnecting parts that come into contact with the cell culture container are sterilized, and manipulations of the cell culture container and the interconnecting parts is minimized and occurs in an aseptic environment, such as a hood or a laminar air flow enclosure, the risk of contamination is reduced. Additional materials and methods that decrease the risks of contamination will be discussed below.

Multi-layer cell culture containers must allow for entry and exit of cells and cell culture media into and out of the cell culture chambers. There is a need to facilitate the movement of fluids into and out of multi-layer cell culture containers in a way that maintains sterility and minimizes spills, while also minimizing the footprint of the multi-layer flask. Minimizing the footprint of these flasks and containers allows for increased utilization of space in incubators, as well as in storage and shipping. In addition, there is a need to provide these multi-level cell culture containers with features that support automated or robotic processes.

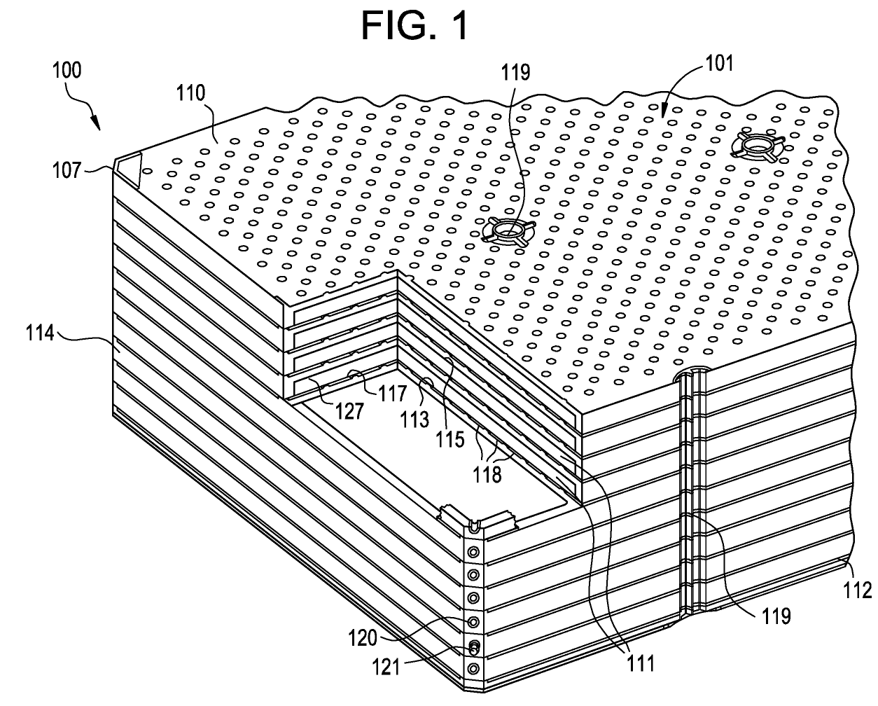

In embodiments of the present invention, a multi-layer flask is provided. An embodiment of the multi-layer flask 100 of the present invention is illustrated in the partial cut-away perspective view shown in FIG. 1. The multi-layer flask 100 has an outer vessel body 101 defined by a top plate 110, a bottom tray (not shown), sidewalls 112, and end walls 114. Disposed within the flask 100 are individual cell growth chambers 111 as can be seen more clearly in the cut-away portion of FIG. 1. The individual cell growth chambers 111 are each defined by a bottom surface 113 and a top surface 115. The surfaces 113 and 115 are attached to the flask body 101 along the sidewalls 112 and end walls 114. Preferably, at least one bottom surface 113 within each chamber 111 is a gas permeable, liquid impermeable material capable of providing a surface for the growth of cells 117. The gas permeable, liquid impermeable material may provide the surface upon which cells attach, or the floor of the cell growth chamber, or it may be the opposite surface, or the ceiling of the cell growth chamber. The bottom surface 113, or the cell culture surface 113 may be flexible or rigid. Each top surface 115 is preferably a rigid, generally gas impermeable material that will provide support to the cell growth chamber 111. The surfaces of the multi-layer flask may be clear, opaque, colored or colorless. In an embodiment of the present invention, there are tracheal spaces 118 between each cell growth chamber 111. The opposing top surface 115 of the chamber 111 defines an upper wall to the cell growth chamber 111 as well as a bottom portion of a tracheal chamber 118. The tracheal chamber 118 is therefore inclusive of a gas permeable, liquid impermeable surface 113 of a first cell growth chamber and an opposing surface 115 of a second growth chamber 111. Supports 119 may also be present to provide structural support to integrally incorporate the surfaces 113 and 115 in forming growth chambers 111 in alternation with tracheal air spaces 118 within the unitary flask 101. Each cell growth chamber 111 therefore alternates with a tracheal chamber 118 in vertical successive orientation.

In one embodiment of the present invention, the individual cell growth chambers 111 permit cellular growth on gas permeable membranes 113 such that multiple cell growth chambers 111 are integral with the body 101 of the multi-layer flask 100 and are capable of being completely filled with nutrient media for the growth of cells. The series of tracheal air spaces 118 through the multi-layer flask 100 provide gaseous communication between the cells 117 growing on gas permeable surfaces 113, in media 127 in the individual cell growth chambers 111 inside the multi-layer flask, and the external environment. The tracheal spaces 118 allow oxygenation of media located within cell growth chambers 111 through the gas permeable surfaces 113. Further, the tracheal chambers 118 may take the form of any air gap or space, and do not allow entrance of liquid. As a result, a rigid cell culture multi-layer flask 100 having multiple growth chambers 111, alternating with tracheal spaces 118, is cooperatively constructed to afford the benefit of equivalent gaseous distribution to a large volume of cells 117.

Gas permeable membrane 113 can be affixed to supports 119 and side walls 112 by any number of methods including but not limited to adhesive or solvent bonding, heat sealing or welding, compression, ultrasonic welding, laser welding and/or any other method commonly used for generating seals between parts. Laser welding around the circumference of the membrane 113 is preferred to establish a hermetic seal around the membrane region such that the membrane is flush with and fused to the face of the supports 119 such it becomes an integral portion of the interior surface of the multi-layer flask. Once the gas permeable membrane 113 is adhered to the sidewalls and endwalls, the top plate 110 and bottom tray 120 may be joined. The bottom tray 120 and top plate 110 may be injection molded. Various sizes and shapes of the supports 119 may be incorporated to facilitate positioning of the membranous layers 113 for cell culture 117 within the cell culture vessel 100.

Gas permeable, liquid impermeable membranes 113 (see FIG. 1) may be made of one or more membranes known in the art. Membranes typically are made of suitable materials that may include for example: polystyrene, polyethylene, polycarbonate, polyolefin, ethylene vinyl acetate, polypropylene, polysulfone, polytetrafluoroethylene (PTFE) or compatible fluoropolymer, a silicone rubber or copolymer, poly(styrene-butadiene-styrene) or combinations of these materials. As manufacturing and compatibility for the growth of cells permits, various polymeric materials may be utilized. For its known competency, then, polystyrene may be a preferred material for the membrane (of about 0.003 inches in thickness, though various thicknesses are also permissive of cell growth). As such, the membrane may be of any thickness, preferably between about 25 and 250 microns, but ideally between approximately 25 and 125 microns.

The multi-layer flask 100 of the present invention may be made by any number of acceptable manufacturing methods well known to those of skill in the art. In an embodiment of a method, the multi-layer flask 100 is assembled from a collection of separately injection molded parts. Although any polymer (such as polystyrene, polycarbonate, acrylic, polystyrene, or polyester) suitable for molding and commonly utilized in the manufacture of laboratory ware may be used, polystyrene is preferred. Although not required, for optical clarity, it is advantageous to maintain a thickness of no greater than 2 mm. The separate parts may be assembled by any number of methods including but not limited to: adhesive or solvent bonding, heat sealing or welding, compression, ultrasonic welding, laser welding and/or any other method commonly used for generating seals between parts such that it becomes an integral portion of the interior surface of the multi-layer flask. The top plate 110 and bottom tray may be aligned and joined, such as by laser welding.

In an embodiment, parts are held together and are adhesive bonded along the seam, ultrasonically welded, or laser welded, bonded using heat platens or by any other methods. Preferably, laser welding equipment is utilized in a partially or fully automated assembly system. The top plate and tray are properly aligned while a laser weld is made along the outer periphery of the joint.

Advantageously and in order to enhance cell attachment and growth, the surfaces internal to the multi-layer flask 100, including the membrane layer, may be treated to enable cell growth. Treatment may be accomplished by any number of methods known in the art which include plasma discharge, corona discharge, gas plasma discharge, ion bombardment, ionizing radiation, and high intensity UV light.

In an alternative embodiment, an individual cell growth chamber may be bounded on one side by a layer of gas permeable membrane 110, attached in a liquid impermeable manner to sidewalls 112 and on another side by a top surface that is a rigid layer, to provide a more rigid element to the individual cell culture growth chamber 111 and the multi-layered flask as a whole. For example, an individual cell growth chamber 111, bounded on a top side by a rigid layer 115, on its edges by sides, and on a bottom side by a gas permeable membrane. This individual cell growth chamber 111 can be stacked on top of another such individual cell growth chamber 111, where the top portion of a rigid layer 115 of one individual cell growth chamber 111 forms a support structure that defines tracheal spaces underneath a gas permeable membrane 113 of the adjacent individual cell growth chamber. In an embodiment, individual cell culture chambers can be assembled into a larger multi-layer cell culture vessel. These individual layers can be snapped together, or otherwise attached to each other using any attachment method known in the art.

FIG. 1 illustrates alternating layers of tracheal air spaces 118 and individual cell growth chambers 111 which form the interior of flask 100. The individual cell growth chambers 111 are defined by liquid impermeable, gas permeable membranes 113 attached in a liquid-impermeable manner to the sidewalls and endwalls of the cell culture vessel. Cell growth media 127 is contained between the membranes 113 and cells grow on the liquid-surface of these membranes 113. In this embodiment, the cell growth chamber 111 may be formed by two layers of gas permeable membrane attached in a liquid impermeable manner to sidewalls 112 to form an individual cell growth chamber 111. Tracheal air spaces 118 form layers between the gas permeable membranes, forming air pockets to allow the gas permeable membranes 113 to exchange air into the cell growth media 127. In this embodiment, tracheal air spaces are supported by supports 119 which separate and support the layers of gas permeable membrane 113 which form individual cell growth chambers 111. An advantage of this embodiment of the multi-layered flask that is compatible with an embodiment of the manifold of the present invention is its enhanced capacity to grow cells on an opposing surface when the multi-layer flask is rotated 180°. Thus, when the multi-layer flask is rotated, cells can be cultured on an alternate gas permeable membrane surface 113. Where only gas permeable membranes are layered intermediary to the multi-layer flask, cell growth is therefore enabled on both of its gas permeable surfaces 113. The membrane 113 allows for the free exchange of gases between the individual cell culture growth chamber 111 and tracheal spaces 118. A preferred embodiment would include a membrane 113 that is additionally durable for manufacture, handling, and manipulation of the multi-layer flask.

Accessibility to the cellular growth chambers 111 is achieved through ports 120 that extend through an external surface of a cellular growth chamber, to create an opening or a pass-through space in a surface of a cellular growth chamber 111. While the port 120 is shown extending through a sidewall in FIG. 1, the port 120 may be located on an endwall or on any other surface of the cellular growth chamber 111. In embodiments of the present invention, each cellular growth chamber 111 has at least one port 120 allowing access into each cell culture chamber. In embodiments, more than one port 120 may be present in each cell culture chamber to allow fluid to enter a cell culture chamber 111 through one port 120 while displaced gas exits the cell culture chamber through another port 120. A port cover 121 is shown in FIG. 1. In this embodiment, the port cover is a plug. However additional embodiments of the port cover will be discussed and disclosed below. In embodiments, port cover 121 may have a septum to prevent contamination of the contents of the cell culture chambers. In embodiments, ports 120 may be treated with a layer of material to improve the water-tight capabilities of the ports. These materials may allow be more amenable to forming liquid-tight seals with cannula introduced into them, or with port covers. Examples of these materials include rubber, PVC, Teflon®, cork, silicone, gum, urethane, or any other material known in the art.

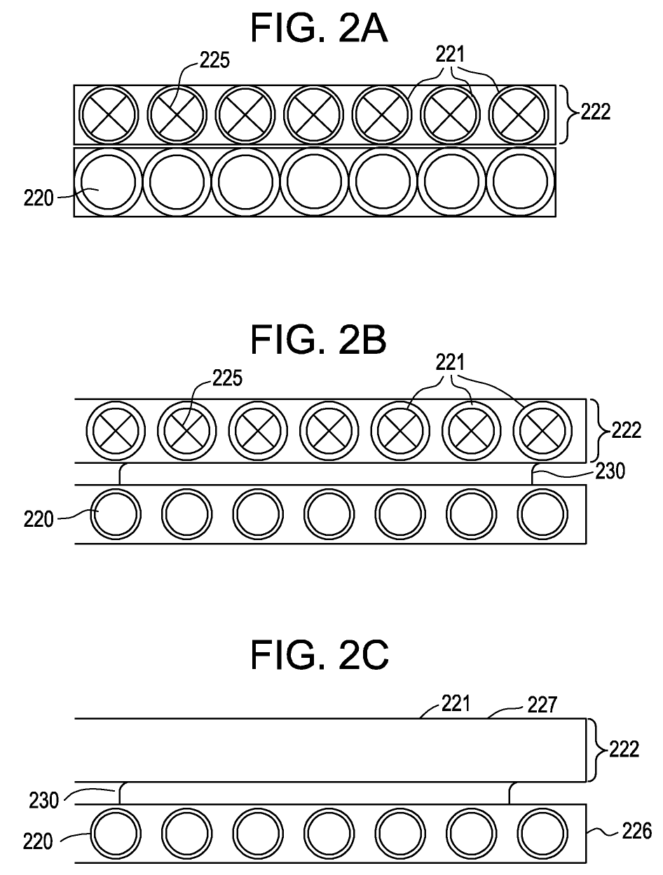

FIG. 1 illustrates an embodiment of the present invention having a set of aligned ports 120, on a corner 107 of the multi-layer flask 100. The corner 107 may be flat, as shown in FIG. 1, or rounded, or any shape. FIG. 1 also illustrates port cover 121 which provide a releasable closing on the port 120. Port cover 121 may be individual port covers 121, as shown in FIG. 1, or may be in strips, as shown in FIG. 2, structured and arranged to close a strip of ports.

FIGS. 2A, 2B and 2C illustrate embodiments of ports and port covers of the present invention. As shown in FIG. 2A-2C, ports 220 may be structured and arranged to reversibly engage with port covers 221 to form a water-tight seal. Port 220 and port cover 221 may form a water-tight seal by forming a friction seal between the parts. Port covers 221 may be in strips 222 structured and arranged to form liquid-tight seals against a plurality of ports 220. Ports 220 may be openings that are flush with the surface of the multi-layer flask or may be raised structures, extending from the surface of the multi-layer flask, defining an opening. These raised structures may be protruding structures that provide a male coupling structure to enable the port to couple with a female port cover or a female fluid flow device. Or, the port may be a female structure to provide coupling structure for a male port cover or a male fluid flow device.

As illustrated in FIG. 2A, port covers 221 may fit within ports 220 to reversibly engage with ports 220 to form liquid-tight seals. Or, as illustrated in FIG. 2B, ports 220 may fit within port covers 221 to reversibly engage to form liquid-tight seals. In an additional embodiment, as illustrated in FIGS. 2A and 2B, port cover 221 may contain septa 225. In additional embodiments, port 220 may contain septa 225 (not shown).

In embodiments, as illustrated in FIG. 2C, ports may be surrounded by a port wall 226, a raised portion surrounding the ports. In this embodiment, the port cover 227 may reversibly engage with port wall 226 to form a liquid-tight seal. Port cover 227, made from flexible plastic, may fit snugly inside port wall 226, or may fit snugly to form a friction fitting outside port wall 226, to form a reversible liquid-tight seal. In embodiments, the port cover 221 may be attached to the multi-layer flask by a connector 230.

In the embodiments shown in FIGS. 2A-2C, ports 220 may be open or sealed by a port cover 221 or 226. Or, ports 220 may be open, and may contain a septum 225. Multi-layer flasks 100 of the present invention may be manufactured, sterilized, packaged into sterile packaging and transported with open ports 220. When they are ready to be used, a multi-layer flask 100 may be placed into an aseptic environment, such as a hood or laminar air flow enclosure, and its sterile packaging may be opened. Liquid may be introduced into the multi-layer flask using a fluid flow device, a device for directing fluid from one place to another, which may include needles, cannula or pipettes, pipette tips, tubes, lines, channels, pipes, ducts, conduits, or any other fluid flow devices known in the art. Once the flask 100 is filled, a port cover 221 or 226 may be placed over the open ports 220 to seal them, before the sealed multi-layer flask is introduced into an experimental environment, such as an incubator. The port cover 221 or 226 may be provided in separate sterile packaging, or may be included in the sterile packaging that contained the multi-layer flask.

Or, in an alternative embodiment, the multi-layer flask of the present invention may be manufactured and sterilized, and the ports may be sealed with a sterile port cover 221 or 226. The sealed sterile multi-layer flask may then be packaged and transported. When ready to be used, the sealed flask may be placed into an aseptic environment such as a hood or laminar flow enclosure, removed from its packaging, and liquid may be introduced into the flask either by opening the ports by removing the port covers, or by introducing liquid into the flask by inserting a needle, cannula, pipette tip, or other device through septa 225 within the port cover 221, and introducing liquid into the multi-layer flask.

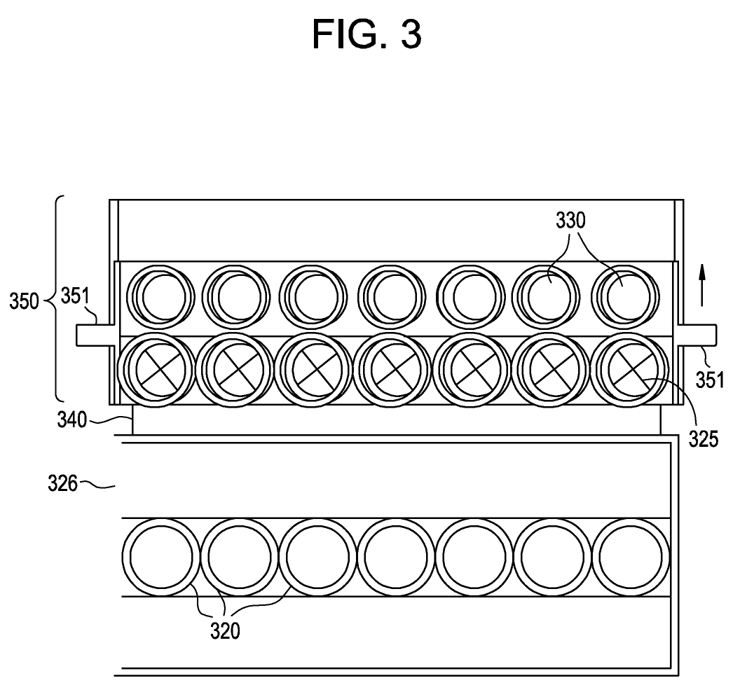

FIG. 3 illustrates another embodiment of a port and port cover of the present invention. FIG. 3 illustrates ports 320 and a sliding port cover 350. The sliding port cover 350 engages with the ports 320, or a port wall 326, to form a releasable water-tight seal. In this embodiment, the sliding port cover 350 has a sliding mechanism to allow the user to choose the desired port cover by sliding the handle 351, as indicated by the arrow. As illustrated in FIG. 3, the sliding port cover 350 allows the user to choose whether the ports will be covered by open or closed, or covered by septa 325, or filters 330. Filters 330 may be filter materials known in the art to reduce or prevent contamination of cells in culture. The sliding port cover may be set in an open position, allowing for the free flow of air into and out of the cell culture chamber. In this open position, the port may be covered with a filter to allow for the flow of air into and out of the cell culture chamber, but prevent contamination. Or, the sliding port cover may be set in a closed position, where the closed position has a septum, to allow a fluid flow device such as a needle or a pipette tip to enter the cell culture chamber through the closed port cover. For example, the sliding port cover 350 may be set to cover the ports 320 with septa 325 by sliding the sliding port cover in the direction of the arrow shown in FIG. 3, until septa 325 are aligned with ports 320. A user may introduce liquid through the ports 320 by inserting a needle or cannula or pipette tip through the septa 325. The septa 325 may be structured or arranged to accommodate a liquid-handling device such as a small or large bore needle, a cannula, or a pipette tip. Once liquid has been introduced through ports 320, the user may choose to leave the ports covered with septa 325, or the user may choose to cover the ports 320 with filters. If the user chooses to cover the ports 320 with filters 330, the user would slide the slide handle in the opposite direction of the handle, to align the filters 330 with the ports 320. In this way, the user may change these coverings by sliding the handle of the port cover from one position to the other. In this embodiment, the sliding port cover 350 may be integral with the multi-layer flask of the present invention, or the sliding port cover may attached to the multi-layer flask by a connector 340. The sliding port cover may be removable. If the sliding port cover 350 is removable, it may be attached to the multi-layer flask by any connector 340 such as a hinged connector 340.

When a port cover contains a septum, and the port cover is engaged with the port to form a reversible liquid-tight seal, the septum may be situated above the port. That is, when the port cover contains a septum, the septum itself may be seated on top of or above the port.

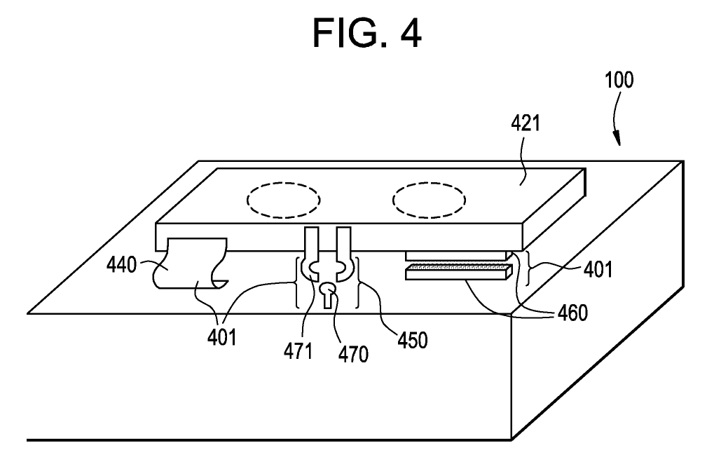

Turning now to FIG. 4, FIG. 4 illustrates embodiments of connectors 401 of the present invention. Connector 401 is a feature which connects the port cover 421 to the multi-layer flask. The connector 401 may be a hinged connector 440. A hinged connector may be a flexible thin ribbon of plastic that is attached on one side of the ribbon to the port cover and on the other side of the ribbon to the port wall or to the multi-layer flask. This thin flexible ribbon of plastic is deformable, therefore allowing port cover to be attached to the multi layer flask either in an open position (not shown), or in a closed position, as shown in FIG. 4. The hinged connector 440 may be a thicker ribbon of plastic that has been scored to allow the plastic to bend at the score marks. Or, the hinged connector may be a pivot hinge, a spring hinge (to ensure that the port cover is in a closed position or in an open position) or any other type of hinge known in the art. These hinged 440 connectors may be molded separately, or molded as a part of the port cover, and attached to the multi-layer flask by any method known in the art, including the methods for molding and attaching plastic parts as discussed above.

In additional embodiments, the connector may be a ball and socket connector 450. A ball feature 470 may protrude from the port cover or the multi-layer flask, and may reversibly engage with protruding socket features 471 in the opposite surface. The port cover may then be snapped into the ball and socket joint to connect the two pieces. In additional embodiments, the connector may be a hook and loop fastener 460, zip-lock type fastener, adhesive, or other connecting mechanisms.

The port cover 421 may be connected to the multi-layer flask 100 (as shown in FIG. 4), or to a structure of the multi-layer flask 100, such as a port wall 226 (as shown in FIGS. 2A-2C) by a connector 401. Or, in alternative embodiments, port cover 221 or port cover strip 222 may not be attached to the multi-layer flask but may be separate from the multi-layer flask 100. Port cover 221 or port cover strip 222 may be disposable, so that each time the cell growth chambers are accessed through ports 220, the used port cover 221 or port cover strip 222 is removed and discarded, and a new port cover 221 or port cover strip 222 is applied to close the ports 220 when the user wishes to close the multi-layer flask. Port cover or port cover strip may be disposable and sterilizable by heat sterilization or UV sterilization, or any other method known in the art.

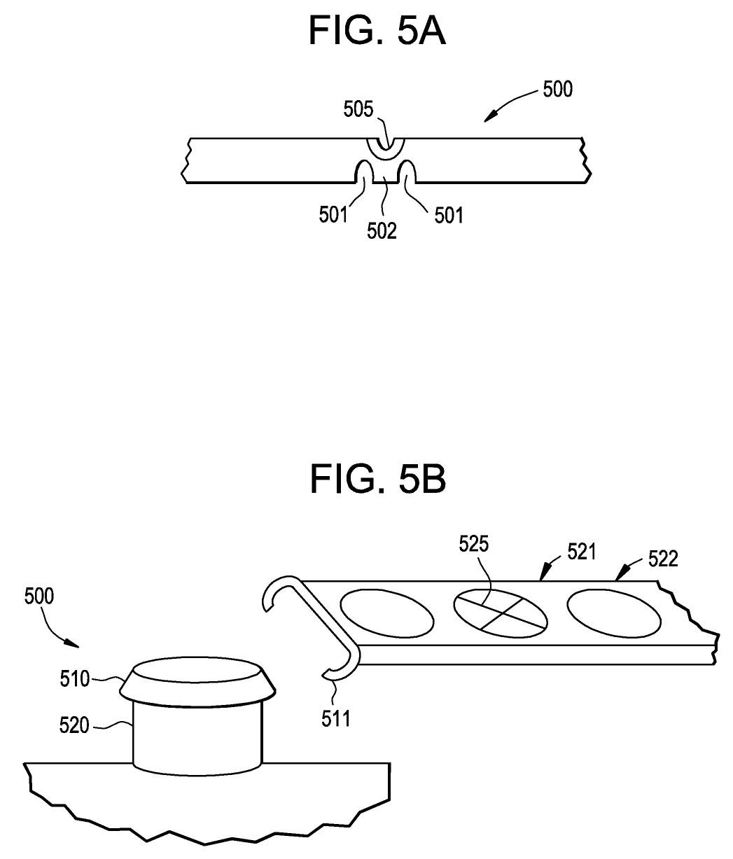

In embodiments, ports 220 and port covers 221 or 226, may have internal or external sealing or engaging structures to allow the port covers to engage against the ports to provide a liquid-tight seal. See for example, FIGS. 5A and 5B. FIG. 5A illustrates a zip-lock type sealer 500. A pair of spaced, parallel fastener ridges 501 forming a channel 502 may be provided on one surface, for example the top surface of a port or a port cover, or a port wall, and the complimentary surface may have a single ribbon of flexible material 505 that, when pressed into the fastener channel, forms a releasable liquid-tight seal. In embodiments, the fastener may be designed to change color when the seal is formed. For example, the fastener ridges may carry a color, for example blue, and the ribbon may carry another color, for example red, and when the two complimentary elements are pressed together to form a seal, a purple color may show through the structure.

FIG. 5B illustrates an alternative embodiment of a sealer 500. A ledge structure 510 or annular structure around the raised port 520 engages with complimentary structures which may be, for example, a flexible catch bar 511 on the port cover 521 or port cover strip 522 to allow the port cover 521 to slidingly engage with the ports to create a releasable liquid-tight seal between the port cover strip 522 and the port(s) 520. While only one port 520 is shown in FIG. 5B, the port cover strip can be used to slidingly engage with a series of similarly structured ports to form seals between a series of ports 520 and a port cover strip 522. These structures taken together, the ledge structure 510, which slidingly engages with the port cover strip 522 to form a liquid-tight seal, are an embodiment of a sealer 500. Or, in additional embodiments, port cover 521 may have internal or external sealers, structures to allow the port covers 521 to engage against the ports 520 to provide a liquid-tight seal. As shown in FIG. 5, port cover 521 may slide onto port 520 to fit on top of port 520 to form a liquid-tight seal. This places a septum 525, contained in the port cover 521, above the port 520, but engaged with the port to form a liquid-tight seal. Port 520 or port cover 521 may have a septum 525. If the septum 525 is in the port cover 521, the septum 525 does not extend down into the port, but is above the port and outside the port. The septum 525 allows the port to be closed unless a needle or cannula or other device is inserted through the septum 525 into the port or port cover. When a needle or cannula or other device is inserted through the septum 525 into the port 520 or port cover 521, the needle or cannula can pass through the port or port cover to allow access into the cell culture chamber of the multi-layer flask, while maintaining a liquid-tight seal between the port or port cover and the needle or cannula.

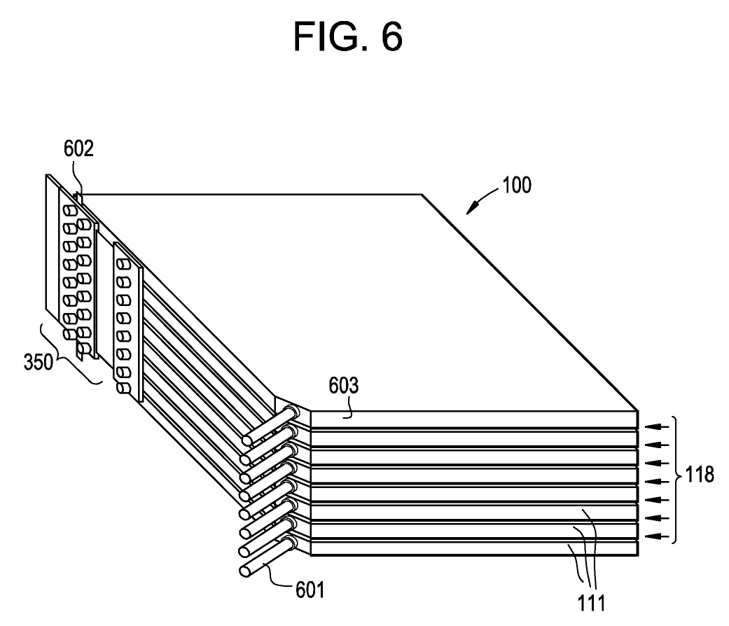

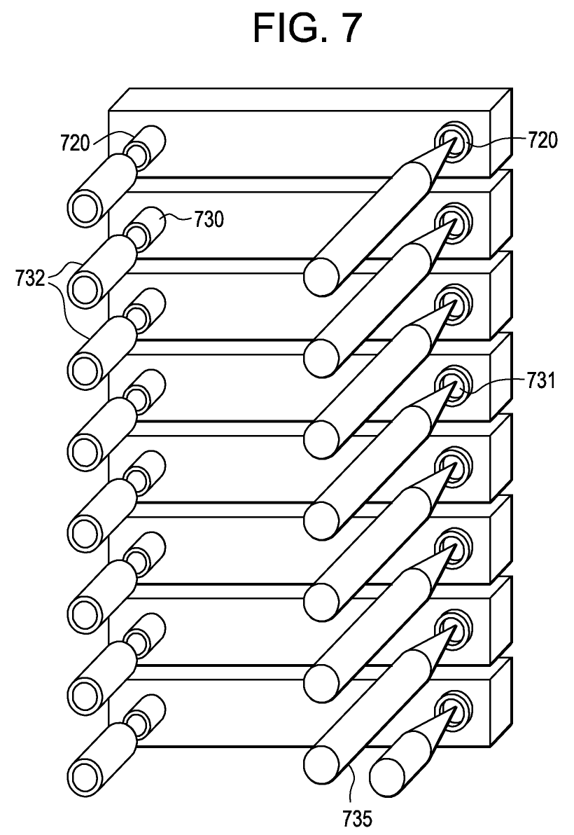

Turning now to FIG. 6, FIG. 6 illustrates an embodiment of the multi-layer flask 100 of the present invention, incorporating the sliding port cover 350 shown in FIG. 3 on a first end 602 of the multi-layer flask 100, and tubes 601 engaged with ports (not shown) on a second end 603 of the multi-layer flask 100. The multi-layer flask has multiple cell growth chambers 111 and integral tracheal air spaces 118. Each cell growth chamber 111 can be accessed through a port (see FIG. 7). In an embodiment of the present invention, a multi-layer flask 100 can be filled with fluid by pumping fluid into each cell growth chamber 111, through a tube 601. For example, filters may be placed over the ports on the first end 602 of the multi-layer flask 100, by sliding the sliding port cover to the filter position, as shown in FIG. 3. Fluid can then be pumped into each cell growth chamber 111 through a tube 601. Air or other gas, displaced by the fluid entering the cell growth chambers 111 through the port on the second end 603 of the multi-layer flask 100, can exit the cell growth chambers through the filtered port on the sliding port cover 350. Once the multi-layer flask is filled, the sliding port cover 350 may be adjusted to cover the ports on the first end 602 of the multi-layer flask with septa to form a liquid-tight seal. Tubes 601 may be sealed by clamping or by welding the tubes closed. The sealed multi-layer flask may then be placed into an appropriate environment, an incubator for example, to allow cells to grow in the multi-layer flask. In an alternative embodiment, fluid may be pumped into a set of cell growth chambers 111 through a first set of tubes 601, may flow through cell growth chambers 111, and may exit cell growth chambers through a second set of ports, coupled to a second set of tubes or other fluid flow devices.

Tubes 601, attached to the multi-layer flask may allow for more sterile transfer of fluid into and out of the multi-layer flask. Tubes can be coupled to additional tubes or other fluid-flow devices using couplers such as male or female structures which accommodate tubes in friction connections to connect a tube to another tube or another structure. In additional embodiments, tubes can be heat-welded together to form uninterrupted sterile fluid flow devices. For example, fluid flowing through heat-welded tubes may connect a multi-well flask to a source or sink of fluid that may be a distance away from the multi-layer flask. To remove or interrupt the connection, tubes need only be cut or folded and clamped or heat welded closed. The multi-layer flask illustrated in FIG. 6 may be assembled to include tubes, sterilized, packaged and shipped so that a user may open sterile packaging, heat-weld the tubing to connect the multi-layer flask to a sink or source of fluid, and use the flask. This flask configuration may decrease the risk of contamination by removing potentially contaminating liquid-handling features such as valves and couplers.

FIG. 7 represents an expanded view of the port area and illustrates connections between ports and tubes or cannula in embodiments of the present invention. Ports 720 can have male 730 or female 731 structures. Male ports 730 can have protruding structures which allow needles, pipette tips, tubes, 732 cannula, or other fluid-flow devices to fit snugly around the male port 730 to form a liquid-tight seal. Female ports 731 can have receptacle structure to allow cannula 735 needles, tubes or pipette tips or other fluid flow devices to fit snugly into the female port 731 to form a liquid-tight seal.

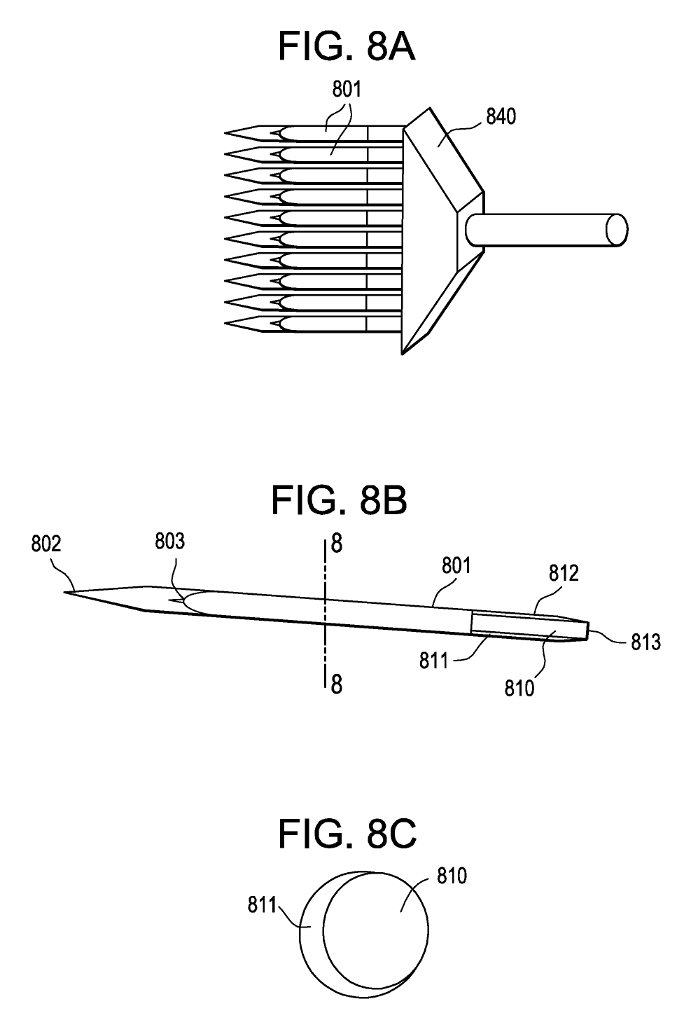

Fluid flow devices, include needles, cannula, pipette tips or tubes and are devices which allow fluid to be directed into and out of a multi-layer flask. FIGS. 8A-8C illustrate one such fluid flow device, a cannula 801. FIG. 8A illustrates multiple cannula 801, attached to a manifold 840. The cannula may be attached to the manifold at their proximal ends 813. In an embodiment, a cannula 801 of the present invention may allow for the directional flow of liquid, while also allowing for a separate flow, or venting of air or gas. For example, the cannula shown in FIG. 8 has a sharp distal tip 802 for piercing a septum and a second tip 803 which is proximal to the first tip. The second tip 803 may also be sharp, and is associated with a second flow path 811. When examined in cross-section as seen in FIG. 8C, which is a cross-sectional illustration, taken at the line 8-8 shown in FIG. 8B, the cannula has two separate flow paths, one for liquid 810 and one for air 811. When the distal end of the cannula 802 is inserted into a port of a multi-layer flask as shown in FIG. 7, liquid can flow into the multi-layer flask through the liquid path 810, and displaced air can escape from the multi-layer flask through the same cannula through the air path 811, which vents to the outside air through air vent 812 which may be located at the proximal end 813 of the cannula. This air vent may be covered with filter material to prevent contamination from this air path. Using this cannula embodiment, liquid can be introduced into a closed cell culture chamber, and displaced air can be vented out at the same time, without the need for a second open port in each cell culture chamber to allow for the release of displaced air. The cannula can be a rigid tubular structure defining a first interior path structured and arranged to conduct fluid and a second interior path structured and arranged to conduct fluid and having a proximal and a distal end.

These fluid flow devices may be coupled to a manifold. A manifold is a device structured and arranged to hold multiple fluid flow devices. The manifold may also be structured and arranged to direct fluid into and out of multiple fluid flow devices. For example, the manifold may be structured and arranged to hold multiple cannula or tubes or pipette tips that are spaced in a way to ensure that the multiple fluid flow devices align with ports of a multi-layer cell culture structure. The manifold may be internal (integral with the multi-layer cell culture structure) or external (separate from the multi-layer cell culture structure).

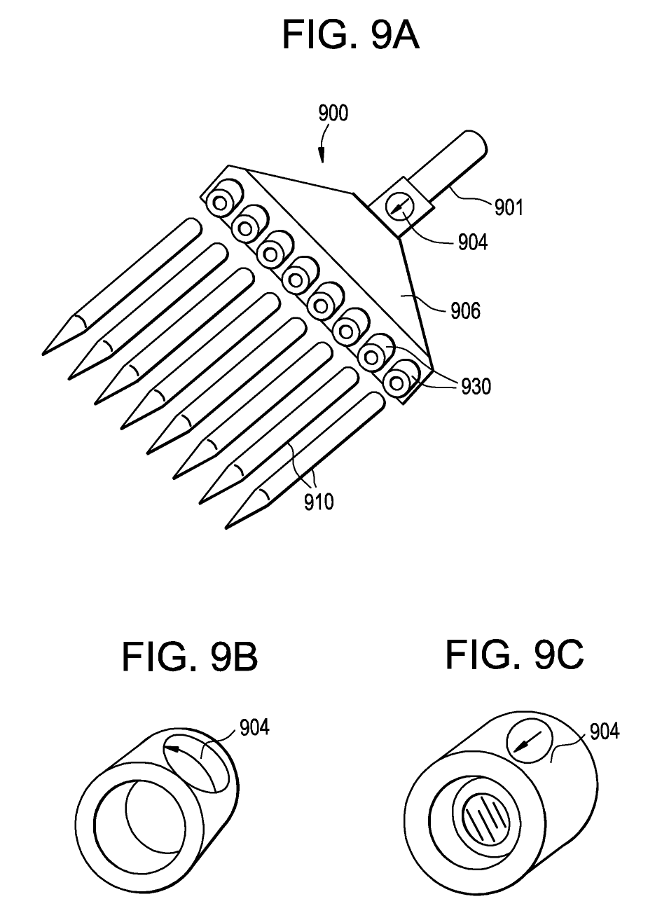

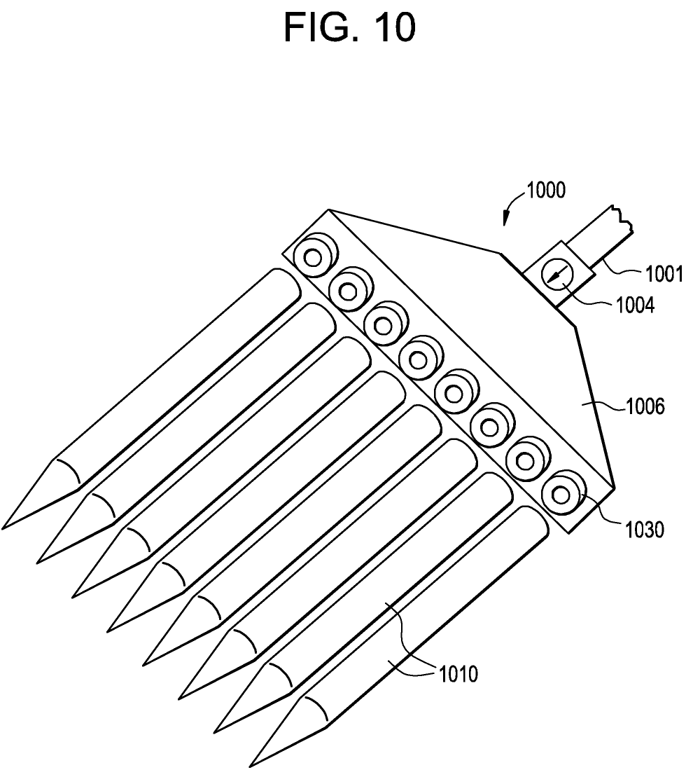

FIG. 9A illustrates an embodiment of an external manifold 900 of the present invention. In an embodiment, the manifold 900 is a device for the manipulation of flask contents as they enter and exit the multi-layer flask. The external manifold 900 has couplers 930 to couple fluid flow devices or cannula 910 to the manifold 900. These couplers 930 may be female structures (as shown), and fluid flow devices such as pipette tips or cannula may insert into these female structures to couple the fluid flow devices to the manifold. Or, the couplers 930 may be male couplers as shown in FIG. 10.

FIG. 10 illustrates an additional embodiment of the external manifold 1000 of the present invention. The manifold 1000 shown in FIG. 10 has a necked opening 1001, a valve 1004 interposed between the necked opening and the manifold body 1006, male couplers 1030 structured and arranged to couple with fluid flow devices 1010 such as cannula, pipette tips or tubing.

Fluid flow devices such as pipette tips or cannula may be structured and arranged to insert into the cell growth chambers through the ports 120 of the multi-layer flask 100. For example, fluids entering the manifold through the necked opening 1001 of the manifold may flow into the manifold body 1006, and through the cannula 1010. When these cannula 1010 are inserted into the ports 120 of the multi-layer flask 100 (see FIG. 1), fluid can flow through the cannula 1010 into the interior of individual cell culture chambers.

Referring again to FIG. 9, the external manifold 900 may have a valve 904. In an open position, as shown in FIG. 9B, fluid may flow freely through the valve 904 from the necked opening 901 to the manifold body 906. In a closed position, as shown in FIG. 9C, fluid may not pass from the necked opening 901 to the manifold body 906. This valve may be operable by rotating a valve key, accessible from the exterior of the external manifold 900. The external manifold illustrated in FIG. 9 may connect multiple cell culture chambers in a multi-layer cell culture flask in parallel. That is, fluid entering the manifold body 906 through the necked opening 901 may flow through multiple fluid flow devices 901 to enter multiple cell culture chambers at the same time. In an alternative embodiment, external manifolds of the present invention may be structured and arranged to allow for fluid to flow from one cell culture chamber in one multi-layer cell culture device to another cell culture chamber in another multi-layer device through the manifold without mixing with fluid bound for another cell culture chamber. In this alternative embodiment, fluid passes from one multi-layer cell culture device to another in series.

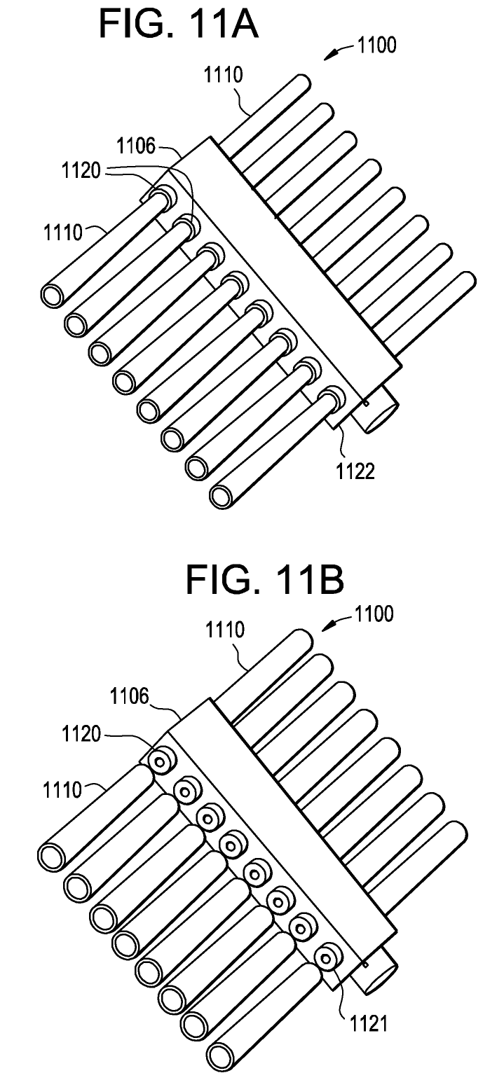

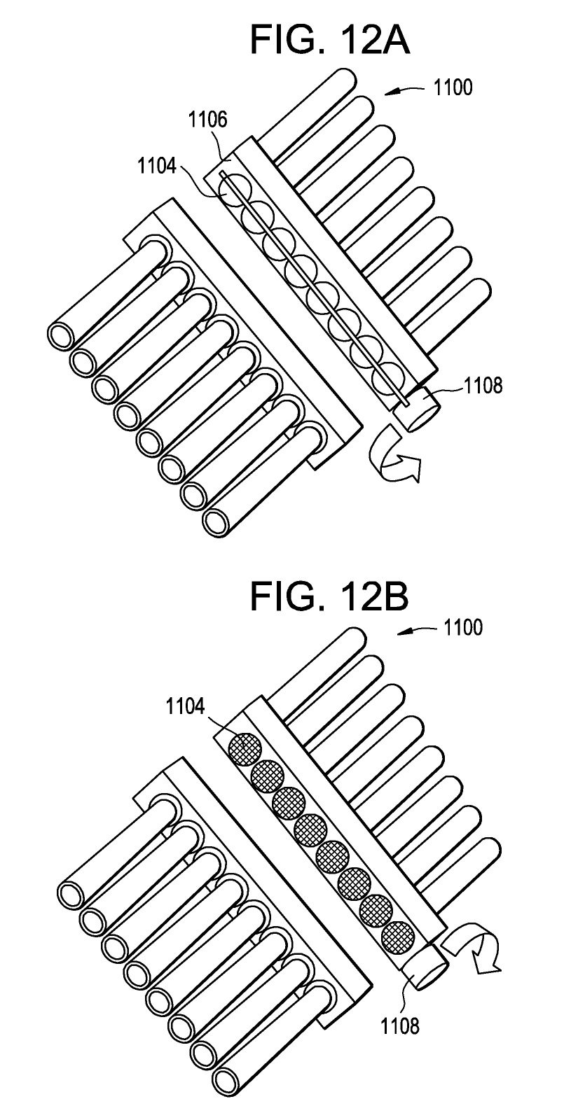

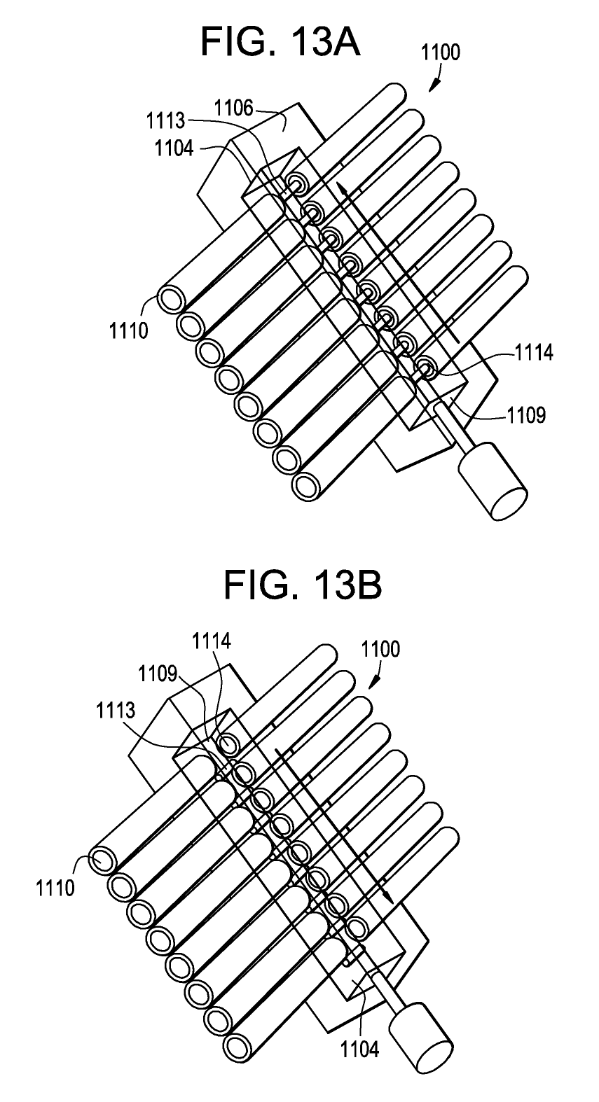

FIGS. 11A and 11B illustrate two embodiments of a series manifold 1100. Fluid flow devices 1110, in this case tubes, are attached to a manifold body 1106. The manifold body 1106 has ports 1120 which are either male 1121 or female 1122 and are structured and arranged to form liquid-tight seals with the tubes 1110. The series manifold 1100 may have a valve to allow fluid to flow from one side of the series manifold to the other side of the series manifold, or to stop fluid from flowing through the manifold body 1106. FIGS. 12A and 12B illustrate an embodiment of a valve 1104 for the series manifold 1100 shown in FIGS. 11A and 11B. In this embodiment, the valve may operate to open (as shown in FIG. 12A) or close (as shown in FIG. 12B) a passageway through the manifold body 1106 from one side of the manifold to the other. A valve opening device 1108 operates to switch the valve between the open and closed position. As shown in FIGS. 12A and 12B, the valve opening device may rotate to open or close the valve as in a butterfly valve. In an alternative embodiment, as illustrated in FIGS. 13A and 13B, the valve opening device 1109 may operate by sliding the valve opening device 1109 from an open position to a closed position, as shown by the arrows. When the valve is open, a passageway 1113 is aligned with openings 1114 on each side of the manifold 1100. When the vale is closed, the passageways 1113 are not aligned with the openings 1114 and no fluid can flow from one side of the manifold to the other. While three embodiments of valves have been particularly illustrated, many valve mechanisms are known in the art and may be applicable in embodiments of the present invention. Valves may be mechanical or electronic, for example solenoid valves or magnetic valves may be used.

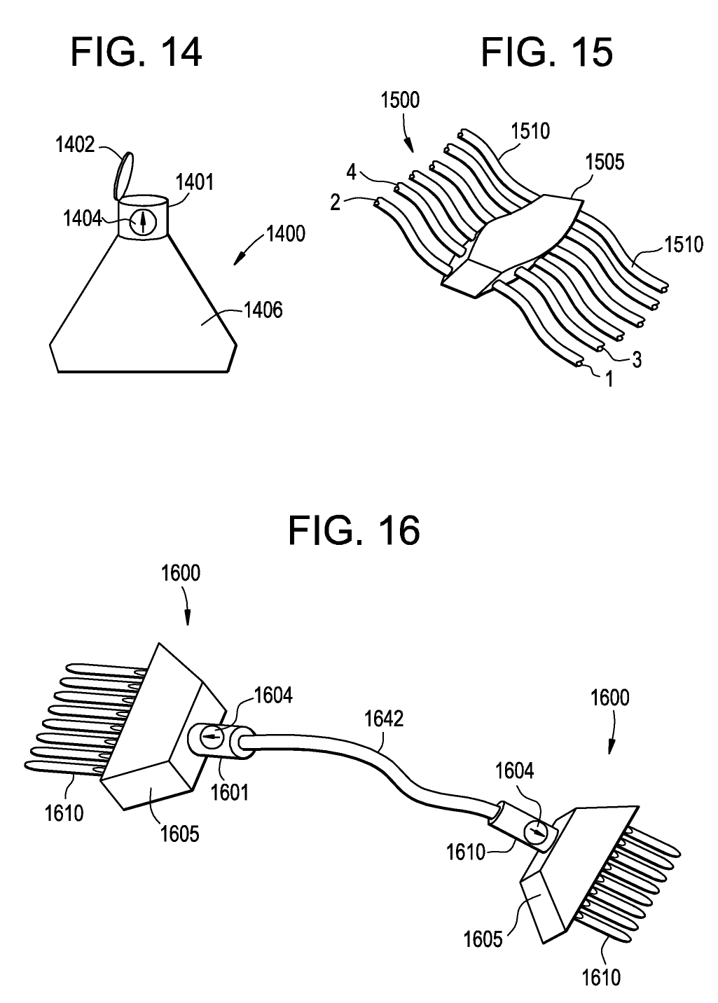

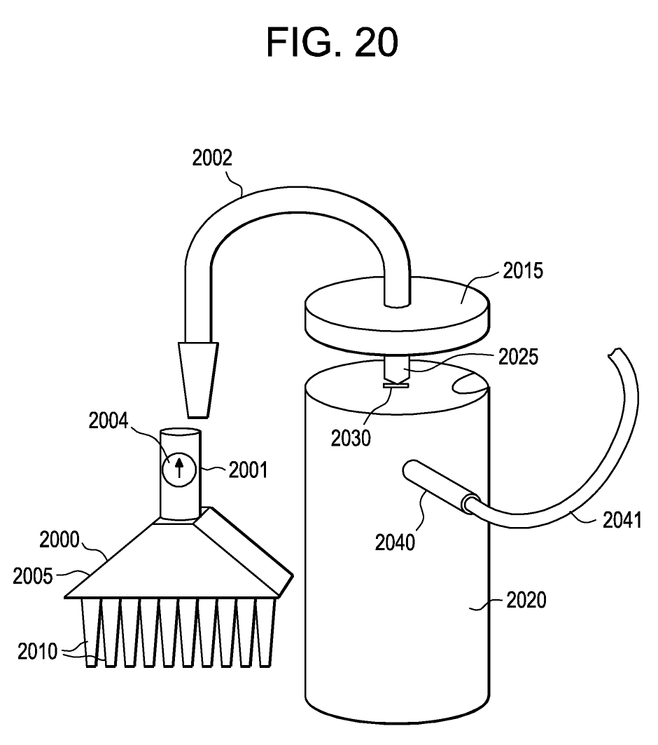

Embodiments of the external manifold are illustrated in FIGS. 14-16. FIG. 14 illustrates an external manifold 1400 having a necked opening 1401, a manifold body 1406 and a valve 1404. The necked opening can be covered by a cap 1402. The cap 1402 may be present or absent. If present, the cap may incorporate filters to allow for the exchange of gas between the internal and external spaces of the cell culture system. In additional embodiments, the necked opening can be attached to tubing which allows for fluid communication from a reservoir of fluid to the manifold, to the multi-layer flask (see FIG. 20).

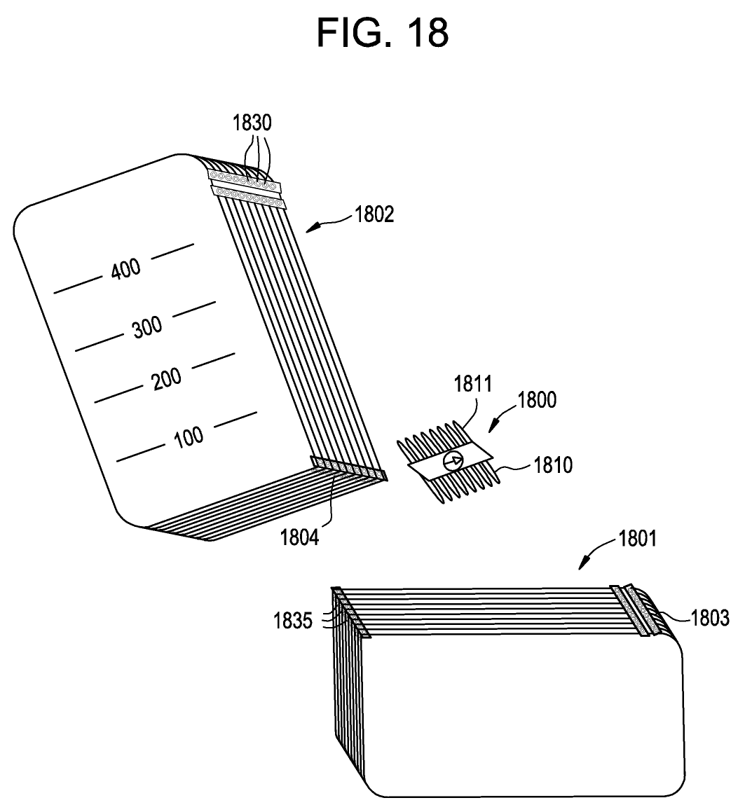

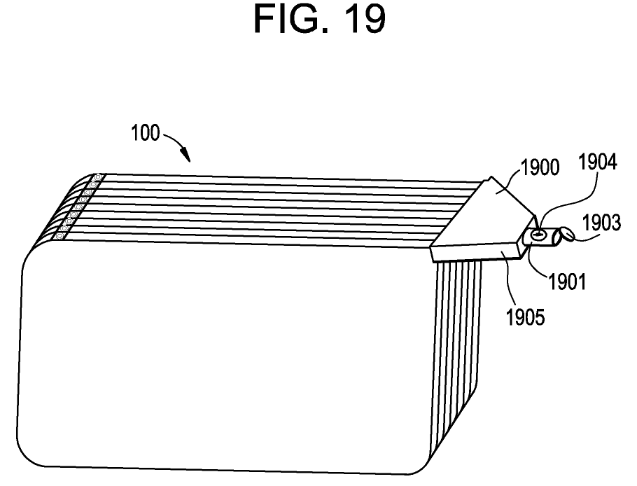

FIG. 15 is an illustration of an additional embodiment of the manifold 1500 of the present invention. In this embodiment, the manifold body 1505 connects two sets of tubes 1510, structured and arranged to insert into two adjacent multi-layer flasks, as shown in FIG. 18. Using this embodiment, it is possible to connect adjacent multi-layer to form a network of multiple multi-layer flasks. In addition, if this manifold embodiment is used, it is possible to fill multiple multi-layer flasks with fluid by administering fluid to one multi-layer flask. For example, two or more multi-layer flasks can be connected together using this manifold embodiment 1500. Fluid can be administered to a multi-layer flask on top of a stack of connected multi-layer flasks. To fill all of the flasks, fluid is allowed to flow from the top flask, through all of the intermediary flasks to a bottom flask until all of the flasks are filled with fluid. Depending upon the internal structure of the manifold, whether fluid is pools in the manifold body 1505, or whether fluid flows from a tube on one side 1510 of the manifold body to a corresponding tube on the other side of the manifold body 1510, fluid can flow through this embodiment of the manifold to multi-layer cell culture flasks in series or in parallel. For example, if the manifold body 1505 allows fluid to mix as it enters the manifold from the cannula, this manifold allows all of the layers within a single multi-layer flask, or between multi-layer flasks to mix, and thereby be connected in parallel. If the manifold body maintains defined connections, it can connect cell culture chambers from flask to flask in series. For example, if tube 1 is connected only to tube 2 through the manifold body, and if tube 3 is connected only to tube 4 as it passes through the manifold body, then the cell culture chamber that is accessed by tube 1 is connected in series with the cell culture chamber that is accessed by tube 2, and the cell culture chamber that is accessed by tube 3 is connected in series with the cell culture chamber that is accessed by tube 4.

In an additional embodiment, tubes 1510 may be replaced by cannula or needles or other fluid flow devices structured and arranged to connect in a liquid-tight manner to the manifold and to the multiple-layer flask. For example, tubing can fit in a friction fit over a port when the port has a height and extends above the surface of the multi-layer flask. Or, in the alternative, tubing may be inserted into a port to form a liquid-tight seal when the port is structured and arranged to form a liquid-tight friction fit between the tubing and the port.

FIG. 16 illustrates an additional embodiment of the present invention. FIG. 16 shows two manifolds 1600 each having a manifold body 1605 connected to cannula 1610, a valve 1604, and a necked opening 1601 connected to tubing 1642 which connects the two manifolds together. It will be understood by those of skill in the art that the manifolds of the present invention may exist in any configuration and may connect one multiple layer flask with another multiple layer flask, may connect a multiple layer flask with a liquid reservoir or a waste container, may connect one manifold with another manifold, and may provide multiple connections through known connectors, valves, pumps or other connections. In addition, it will be understood by those of skill in the art that any of the features described with respect to any of the embodiments disclosed herein may be practiced in different combinations without deviating from the scope of the invention.

In all manifold embodiments, the distance between the cannula can correspond to the distance between the ports of cell culture chambers in the multi-layer flask. The number of cannula present can correspond to the number of layers or cell culture chambers in the multi-layer flask. Embodiments of the manifold may be packaged in-place in a multi-layer flask, to be removed by the user, or may be packaged separately from the multi-layer flask. Manifolds, multi-layer flasks, port covers, port cover strips, tubing and all of the features and accessories described herein may be sterilized and sold in sterile packaging, together or separately.

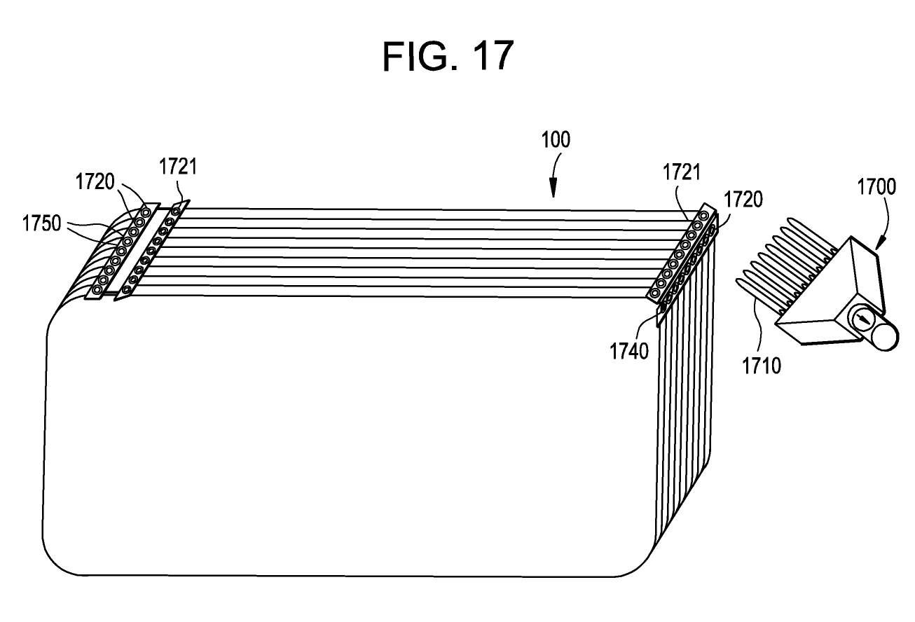

Turning now to FIG. 17, there may be ports 1720 on two sides of the multi-layer flask 100, which may be entry ports 1740 and exit ports 1750. In an embodiment, as fluid enters the multi-layer flask 100 through cannula 1710 of a manifold 1700 inserted into the entry ports 1740, displaced fluid or gas can leave the multi-layer flask 100 through the exit ports 1750 on the other side of the flask. Fluid can be moved through the manifold by providing positive or negative pressure to the manifold, for example by using a pump or a vacuum attached to the manifold or the flask (see FIG. 20). Once the cell culture chambers of the multi-layer flask 100 have been completely filled with fluid, the cannula 1710 of the manifold 1700 can be removed from the entry ports 1740, and both the entry ports 1740 and the exit ports 1750 can be plugged with port covers 1721. The multi-layer flask 100, in this configuration, with both entry and exit ports closed by port covers, is a closed multi-layer cell culture system. This closed multi-layer flask can then be rotated to maximize the cell growth surface within the cell growth chambers, (i.e., to put the bottom plate down) and placed in an appropriate location for cell culture, such as an incubator. In additional embodiments, the manifold 1700 may not have cannula but may form a liquid-tight seal directly with ports (see for example, FIG. 10).

When the time comes to empty the cell culture chamber, the multi-layer flask 100 can be removed from its location for cell growth, rotated so that the ports are in an “up” position, the port covers 1721 on both the entry and the exit ports can be removed, either manually or by robotic manipulation, and the cannula of a manifold can be inserted into the multi-layer flask to remove fluid, either by suction or by gravity. As the multi-layer flask empties, the flask can be tilted so that the remaining fluid is presented to the tips of the cannula, extending into the multi-layer flask 100.

FIG. 18 is a perspective view of an embodiment of the present invention showing how multiple flasks can be connected together by an embodiment of the manifold of the present invention. When the cannula 1810 on one side of the manifold 1800 are inserted into ports 1803 of one multi-layer flask 1801, and the cannula 1811 on the other side of the manifold 1800 are inserted into ports 1804 of the other multi-layer flask 1802, the two flasks can be joined together. Media and cells can be transferred from one vessel to the other through the manifold 1800, so connected. For example, the vessel with cells to be distributed to another vessel (or several other vessels for cell proliferation) can be tilted upward as shown in FIG. 18, and the cells can be transferred from a first vessel 1802 to a second vessel 1801 by gravity. In an alternative embodiment, a pump can be attached to a second set of ports 1830 to drive fluid from the first vessel 1802 to the second vessel 1801. Or, a vacuum pump could be attached to the ports 1835 of the receiving vessel to pull fluid from the first vessel 1802 to the second vessel 1801.

FIG. 19 illustrates an additional embodiment of an embodiment of the manifold, as shown in FIG. 14, attached to a multi-layer flask. FIG. 19 shows a multi-layer flask 100 with a manifold 1900 attached. The manifold 1900 has a manifold body 1905, a necked opening 1901, a cap 1903 and a valve 1904. The manifold body 1905 couples with the multi-layer flask 100 through the ports (not shown) to allow fluid entering the necked opening 1901 to flow through the manifold 1900 and into the cell culture chambers of the multi layer flask 100. The manifold 1900 may be permanently attached to the multi-layer flask, or it may be removable. The manifold 1900 may be disposable, and sterilizable. When the manifold 1900 is removed, the ports of the multi-layer flask may be covered by port covers, and access to the cell culture chambers of the multi-layer flask is limited. When the manifold 1900 is removed, the multi-layer flask has a regular rectangular or square footprint, allowing multiple multi-layer flasks to be placed into an enclosed space such as an incubator or a packing box without the need to accommodate an irregularly shaped manifold or opening.

FIG. 20 illustrates an embodiment of the present invention connected to an embodiment of a reservoir or cell collection device 2020. While the external manifold shown in FIG. 20 is the embodiment shown in FIGS. 8, 9 and 10, any external manifold embodiment may be appropriate here. The external manifold 2000 has cannula 2010, a manifold body 2005, a valve 2004, and a necked opening 2001. The external manifold 2000 is structured and arranged to couple with a multi-layer cell culture container (not shown). In this embodiment, the external manifold may be couples to an external liquid reservoir which can be a cell collection device or a culture media reservoir. Shown in FIG. 20 is a cell collection device 2020. The external manifold 2000 can be coupled to the cell collection device through a container cap 2015, which is attached to the cell collection container 2020 by tubing 2002. The container-end of the tubing 2025 slips into a perforation 2030 in the container 2020 when the container cap 2015 is screwed or snapped into place. In an alternative embodiment, the collection container can be preassembled with the tubing 2002 already attached to the container 2020. The container 2020 has a second port 2040 for connecting to tubing 2041 which leads to a vacuum pump (not shown). When this assembly is connected to a multi-layer flask through the ports of the multi-layer flask, and a vacuum is provided to the container through the second port 2040, the vacuum can cause liquid and, in some cases cells, to be removed from the multi-layer flask and deposited into the container 2020. The container may be a sterile container, and the external manifold and tubing may be sterilized, allowing for the sterile removal of cells and fluid from a multi-layer cell culture flask to a container.

These processes can be performed in an automated setting. For example, an external manifold, connected to a sterile collection container as shown in FIG. 20, may be manipulated robotically to couple to a multi-layer flask and remove the contents of the multi-layer flask. With this kind of robotic manipulation, human contact is reduced and the risks of contamination and spilling are reduced.

The invention being thus described, it would be obvious that the same may be varied in many ways by one of ordinary skill in the art having had the benefit of the present disclosure. Such variations are not regarded as a departure from the spirit and scope of the invention, and such modifications as would be obvious to one skilled in the art are intended to be included within the scope of the following claims and their legal equivalents.

1. A multi-layer cell culture device comprising:
a. at least three cell culture chambers, each cell culture chamber having at least one port, each port having a port cover;
b. at least two integral tracheal chambers;
c. wherein each port is structured and arranged to engage with a hinged port cover to provide a releasable liquid tight seal.

2. The multi-layer cell culture device of claim 1 wherein the port cover is attached to the multi-layer cell culture device by a hinged connector.

3. The multi-layer cell culture device of claim 1 wherein the connector is a hinged connector.

4. The multi-layer cell culture device of claim 1 wherein the port cover comprises a septum.

5. The multi-layer cell culture device of claim 4 wherein the septum is structured and arranged to allow for the introduction of a fluid flow device through the septum to form a liquid-tight coupling between the fluid flow device and the septum.

6. The multi-layer cell culture device of claim 5 wherein the fluid flow device is a needle or a cannula.

7. The multi-layer cell culture device of claim 1 wherein at least one port has a sealer to releasably seal a port cover to the port.

8. The multi-layer cell culture device of claim 7 wherein the sealer comprises an annular feature structured and arranged to engage with a complimentary feature on the port cover to form a liquid-tight seal.

9. A multi-layer cell culture device comprising:
a. at least three cell culture chambers, each cell culture chamber having at least one port;
b. at least two integral tracheal chambers; and,
c. a sliding port cover structured and arranged to engage with the at least one port of the at least three cell culture chambers of the multi-layer cell culture device and to provide an open or a closed port wherein the open or closed access is determined by slidingly engaging the port cover in an open position or a closed position in relation to the at least one port.

10. The multi-layer cell culture device of claim 9 wherein the sliding port cover is connected to the multi-layer cell culture device by a hinged connector.

11. The multi-layer cell culture device of claim 9 wherein the sliding port cover is integral with the multi-layer cell culture device.

12. The multi-layer cell culture device of claim 9 wherein the port cover in the open position comprises a filter.

13. The multi-layer cell culture device of claim 9 wherein the port cover in the closed position comprises a septum.

14. A multi-layer cell culture device comprising:
a. at least three cell culture chambers, each cell culture chamber having at least one port;
b. at least two integral tracheal chambers;
c. a removable manifold structured and arranged to couple with the at least one port of the at least three cell culture chambers;
d. wherein the removable manifold is structured and arranged to form a releasable liquid-tight seal with the at least one port of the at least three cell culture chambers.

15. The multi-layer cell culture device of claim 14 wherein the removable manifold comprises a valve.

16. The multi-layer cell culture device of claim 14 wherein the removable manifold is structured and arranged to form a releasable liquid-tight seal with at least one port of the at least three cell culture chambers of more than one multi-layer cell culture device.

17. The multi-layer cell culture device of claim 14 wherein the removable manifold is structured and arranged to couple the multi-layer cell culture device with a liquid reservoir.

18. A manifold comprising:
a. at least two fluid flow devices structured and arranged to engage with at least two cell culture chambers of a first multi-layer cell culture device; and,
b. at least two fluid flow devices structured and arranged to engage with at least two cell culture chambers of a second multi-layer cell culture device.

19. The manifold of claim 18 wherein the fluid flow device is a cannula, a needle or a tube.

20. The manifold of claim 18 wherein the manifold further comprises a valve.

21. A multi-layer cell culture vessel comprising:
a. at least three rigid cell culture chambers, each cell culture chamber having at least one port;
b. wherein at least one port has a protruding male feature structured and arranged to couple with a female fluid flow device.

22. The multi-layer cell culture device of claim 21 wherein at least one port comprises a female feature structured and arranged to couple with a male fluid flow device.

23. The multi-layer cell culture device of claim 21 wherein the multi-layer cell culture vessel further comprises at least one port cover.

24. The multi-layer cell culture device of claim 23 wherein at least one port cover comprises a septum.

25. A cell culture system comprising:
a. at least one multi-layer cell culture device having at least two cell culture chambers;
b. at least one external manifold having a manifold body and at least two fluid flow devices structured and arranged to handle the flow of fluid between the at least one external manifold and the at least two cell culture chambers of the multi-layer cell culture device;
c. wherein fluid flows into the external manifold and is pooled in a manifold body before being distributed to the at least two fluid flow devices allowing fluid to flow between the at least one external manifold and the at least two cell culture chambers in parallel.

26. A cannula comprising:
a. a rigid tubular structure defining a first interior path structured and arranged to conduct fluid and a second interior path structured and arranged to conduct fluid and having a proximal and a distal end;
b. wherein the distal end has a sharpened tip; and,
c. wherein the proximal end is structured and arranged to engage with a manifold.

27. The cannula of claim 26 wherein the first interior path comprises a first distal end having a sharpened tip and the second interior path comprises a second end wherein the second end is proximal to the first distal end.

28. The cannula of claim 27 wherein the second end comprises a sharpened end.

29. The cannula of claim 26 wherein the second interior path comprises an air vent at the proximal end of the cannula.

30. The cannula of claim 29 wherein the air vent comprises a filter.
